(19) United States
(12) Patent Application Publication
(10) Pub. No.: US 2003/0045747 A1
(43) Pub. Date: Mar. 6, 2003

(54) Method for carrying out a baeyer-villiger oxidation of organic carbonyl compounds

(76) Inventors: Hanns Wurziger, Darmstadt (DE); Guido Pieper, Mannheim (DE); Norbert Schwesinger, Ilmenau (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/221,352

(22) PCT Filed: Feb. 22, 2001
(86) PCT No.: PCT/EP01/02004

(30) Foreign Application Priority Data
Mar. 14, 2000 (DE) 100 12 340.6

(51) Int. Cl. C07C 51/16; C07C 51/23

(52) U.S. Cl. 562/418

(57) ABSTRACT

The present invention relates to a process for the Baeyer-Villiger oxidation of organic carbonyl compounds.

The present invention relates to a process for the Baeyer-Villiger oxidation of organic carbonyl compounds.

The Baeyer-Villiger oxidation of organic carbonyl compounds is a process which is carried out very frequently in the chemical industry and whose great importance is also reflected in numerous publications on this subject.

However, the performance of Baeyer-Villiger oxidations on an industrial scale is associated with safety problems and risks. Firstly, use is frequently made of highly toxic chemical substances, which even alone represent a considerable risk to humans and the environment, and secondly Baeyer-Villiger oxidations frequently proceed highly exothermically, which means that there is an increased risk of explosion when these reactions are carried out on an industrial scale. The attainment of official approval in accordance with the German Federal Emissions Protection Act (BimschG) for the operation of plants for the Baeyer-Villiger oxidation of organic carbonyl compounds on an industrial scale is therefore associated with considerable effort.

The object of the present invention was therefore to provide a novel process for the Baeyer-Villiger oxidation of organic carbonyl compounds which can be carried out in a simple, reproducible manner with increased safety for humans and the environment and with good yields.

This object is achieved in accordance with the invention by the provision of a novel process for the Baeyer-Villiger oxidation of organic carbonyl compounds in which at least one organic carbonyl compound in liquid or dissolved form is mixed with at least one oxidant in liquid or dissolved form in at least one microreactor and reacted for a residence time, and the oxidised organic carbonyl compound is, if desired, isolated from the reaction mixture.

Advantageous embodiments of the process according to the invention are claimed in the sub-claims.

For the purposes of the invention, a microreactor is a reactor having a volume of ≦1000 μl in which the liquids and/or solutions are intimately mixed at least once. The volume of the microreactor is preferably ≦100 μl, particularly preferably ≦50 μl.

A microreactor is preferably made from thin silicon structures connected to one another.

The microreactor is preferably a miniaturised flow reactor, particularly preferably a static micromixer. The microreactor is very particularly preferably a static micromixer as described in WO 96/30113, which is incorporated herein by way of reference and is regarded as part of the disclosure.

A microreactor of this type has small channels in which liquids and/or chemical compounds in the form of solutions are preferably mixed with one another by means of the kinetic energy of the flowing liquids and/or solutions.

The channels of the microreactor preferably have a diameter of from 10 to 1000 μm, particularly preferably from 20 to 800 μm and very particularly preferably from 30 μm to 400 μm.

The liquids and/or solutions are preferably pumped into the microreactor in such a way that they flow through the latter at a flow rate of from 0.01 μl/min to 100 ml/min, particularly preferably from 1 μl/min to 1 ml/min.

In accordance with the invention, the microreactor is preferably heatable.

For the purposes of the invention, the residence time is the time between mixing of the organic carbonyl compound and the oxidant or solutions thereof and work-up of this reaction solution for analysis or isolation of the desired oxidised product(s).

The residence time necessary in the process according to the invention depends on various parameters, such as, for example, the reactivity of the organic carbonyl compounds and oxidants employed or the temperature. It is possible for the person skilled in the art to match the residence time to these parameters and thus to achieve an optimum course of the reaction. The residence time of the reaction solution in the microreactor, where appropriate in the microreactor and the residence zone, is preferably from ≦1 second to ≦15 hours, particularly preferably from ≦1 minute to <3 hours.

In accordance with the invention, the microreactor is preferably connected via an outlet to at least one residence zone, preferably a capillary, particularly preferably a heatable capillary. After mixing in the microreactor, the liquids and/or solutions are fed into this residence zone or capillary in order to extend their residence time.

The reaction mixture is preferably passed through two or more microreactors connected in parallel or in series. This achieves an extension of the residence time, even at an increased flow rate, and the oxidation reaction components employed are converted virtually completely into the desired oxidised organic compound(s).

In a further preferred embodiment of the process according to the invention, the number and arrangement of the channels in one or more microreactors are varied in such a way that the residence zone is extended, likewise resulting in virtually complete conversion into the desired oxidised organic compound(s) at the same time as an increased flow rate.

The residence time of the reaction solution in the system used, comprising at least one microreactor and, where appropriate, a residence zone, can also be set through the choice of flow rate of the liquids and/or solutions employed.

The process according to the invention can be carried out in a very broad temperature range, which is essentially restricted by the heat resistance of the materials employed for the construction of the microreactor, any residence zone and further constituents, such as, for example, connections and seals, and by the physical properties of the solutions and/or liquids employed. The process according to the invention is preferably carried out at a temperature of from −100 to +250° C., particularly preferably from −78 to +150° C., very particularly preferably from 0 to +40° C.

The process according to the invention can be carried out either continuously or batchwise. It is preferably carried out continuously.

For carrying out the process according to the invention for the Baeyer-Villiger oxidation of organic carbonyl compounds, it is necessary for the oxidation reaction to be carried out in the homogeneous liquid phase, since otherwise the channels present in the microreactors become blocked.

The course of the oxidation reaction in the process according to the invention can be followed using various analytical methods known to the person skilled in the art and if necessary regulated. The course of the reaction is preferably followed by chromatography, particularly preferably by gas chromatography, and if necessary regulated.

The isolation of the oxidised organic compound(s) which may be necessary can likewise be carried out by various methods known to the person skilled in the art. The oxidised product(s) is/are preferably isolated from the reaction mixture by extraction, preferably with an organic solvent, or by precipitation, preferably with an organic solvent and/or water, particularly preferably with water.

Organic carbonyl compounds which can be employed in the process according to the invention are all organic carbonyl compounds which are known to the person skilled in the art as substrates of Baeyer-Villiger oxidation reactions.

The organic carbonyl compounds employed are preferably aliphatic, cycloaliphatic, aromatic or heteroaromatic ketones. It is also possible to employ mixtures of various organic carbonyl compounds in the Baeyer-Villiger oxidation process according to the invention, but preferably only one carbonyl compound is employed in each case. The organic carbonyl compounds employed are particularly preferably acetone, cyclohexanone, cyclopentanone or butanone.

Oxidants which can be employed in the process according to the invention are all oxidants which are known to the person skilled in the art for Baeyer-Villiger oxidations. The oxidants can be employed either in pure form or in the form of their mixtures. The oxidants are preferably employed in pure form.

The oxidants employed are preferably inorganic or organic peroxides, hydrogen peroxide, an adduct of hydrogen peroxide and urea, peroxo complexes of transition metals, mixtures of peroxo compounds with organic acids and/or inorganic acids and/or Lewis acids, organic peracids, inorganic peracids, dioxiranes or mixtures of these oxidants.

The inorganic peroxide employed is particularly preferably an ammonium peroxide, an alkali metal peroxide, an ammonium persulfate, an alkali metal persulfate, an ammonium perborate, an alkali metal perborate, an ammonium percarbonate, an alkali metal percarbonate, an alkaline-earth metal peroxide, zinc peroxide or a mixture of these oxidants. The alkali metal peroxide employed is preferably sodium peroxide.

The organic peroxide employed is particularly preferably tert-butyl hydroperoxide, cumene hydroperoxide, menthyl hydroperoxide, 1-methylcyclohexane hydroperoxide or a mixture of these compounds.

The peroxo complexes of transition metals employed are particularly preferably peroxo complexes of the transition metals iron, manganese, vanadium or molybdenum or mixtures of these peroxo complexes. It is also possible here for a peroxo complex to contain two or more identical or different transition metals.

The peroxo compound with an inorganic acid is particularly preferably potassium peroxodisulfate with sulfuric acid, and the peroxo compound with a Lewis acid is particularly preferably hydrogen peroxide with boron trifluoride.

The organic peracid employed is particularly preferably perbenzoic acid, m-chloroperbenzoic acid, magnesium monoperphthalic acid, peracetic acid, peroxytrifluoroacetic acid or a mixture of these peracids.

It is essential for the process according to the invention that the organic carbonyl compounds and oxidants employed are either themselves liquid or are in dissolved form. If these compounds are not already themselves in liquid form, they must therefore be dissolved in a suitable solvent before the process according to the invention is carried out. The solvents employed are preferably halogenated hydrocarbons, particularly preferably dichloromethane, chloroform, 1,2-dichloroethane or 1,1,2,2-tetrachloroethane, paraffins, particularly preferably hexane or ligroin, ethers, particularly preferably diethyl ether, acid amides, particularly preferably N,N-dimethylformamide, nitrites, particularly preferably acetonitrile, carbon disulfide, nitroaliphatic compounds, particularly preferably nitromethane, nitroaromatic compounds, particularly preferably nitrobenzene, or mixtures of the above solvents.

The molar ratio between the organic carbonyl compound and the oxidant employed in the process according to the invention depends, inter alia, on the reactivity of the organic carbonyl compounds employed and the oxidants used. The molar ratio between the organic carbonyl compound and the oxidant is preferably from 1:10 to 1:5, particularly preferably from 1:2 to 1:1.5 and very particularly preferably from 1:1 to 1:1.2.

The risk to humans and the environment caused by escaping chemicals is considerably reduced in the process according to the invention. Furthermore, the risk of an explosion in highly exothermic Baeyer-Villiger oxidations is reduced, inter alia, due to improved mass and heat transport compared with conventional systems. Official approval in accordance with the German Federal Emissions Protection Act (BimschG) for the operation of plants for carrying out the process according to the invention is therefore much simpler to obtain.

It is also particularly advantageous that the process according to the invention can be carried out continuously. This enables the process to be carried out more quickly and inexpensively compared with conventional processes, and it is possible to prepare any desired amounts of the oxidised organic compounds without major measurement and regulation complexity. The course of the Baeyer-Villiger oxidation reaction can be regulated very quickly in the process according to the invention. The oxidation of organic carbonyl compounds by the process according to the invention also enables better control of reaction duration and reaction temperature than is possible in the conventional processes. The temperature can be selected individually and kept constant in each volume element of the system. The oxidised organic products can thus be obtained in very good and reproducible yields.

The invention is explained below with reference to an example. This example serves merely to explain the invention, but does not restrict the general inventive idea.

### EXAMPLE

Baever-Villiger Oxidation of Cyclohexanone to Caprolactone:

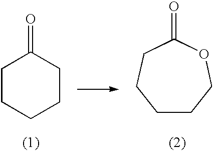

The Baeyer-Villiger oxidation of cyclohexanone (1) to caprolactone (2) was carried out by means of m-chloroperbenzoic acid and trifluoroacetic acid in a static micromixer (Technical University of Ilmenau, Faculty of Machine Construction, Dr.-Ing. Norbert Schwesinger, PO Box 100565, D-98684, llmenau) having a physical size of 0.8 mm×0.8 mm×0.6 mm, and having a total of 11 mixing stages each with a volume of 0.125 μl. The total pressure loss was about 1000 Pa. The static micro-mixer was connected via an outlet and an Omnifit medium-pressure HPLC connector (Omnifit, Great Britain) to a Teflon capillary having an internal diameter of 0.49 mm and a length of 1.0 m. The static micro-mixer and the Teflon capillary were at room temperature.

200 mg (2 mmol) of cyclohexanone were dissolved in 8 ml of CH₂Cl₂. Part of the resultant solution was then introduced into a 2 ml polypropylene disposable syringe. Furthermore, a solution of 860 mg (5 mmol) of m-chloroperbenzoic acid and 150 μl (2 mmol) of trifluoroacetic acid in 8 ml of CH₂Cl₂ was prepared, and part of this solution was introduced into a 2 ml polypropylene disposable syringe.

The contents of the two syringes were subsequently transferred into the static micromixer at a temperature of 30° C. by means of a metering pump (Harvard Apparatus Inc., Pump 22, South Natick, Mass., USA). Various residence times were set via the flow rate, resulting in various yields in the oxidation of (1) to (2). The yields were determined by GC-MS spectrometry in a Hewlett-Packard instrument without prior work-up of the reaction mixture.

The flow rates and the resultant residence times and yields are shown in Table 1 below:

TABLE 1

| Flow rate | Residence time | |
|---|---|---|
| [μl/min] | [min] | (1):(2) ratio |
| 10 | 30 | 82:18 |
| 5 | 60 | 30:70 |
| 2.5 | 120 | 0:100 |

1. Process for the Baeyer-Villiger oxidation of organic carbonyl compounds, characterised in that at least one organic carbonyl compound in liquid or dissolved form is mixed with at least one oxidant in liquid or dissolved form in at least one microreactor and reacted for a residence time, and the oxidised organic carbonyl compound is, if desired, isolated from the reaction mixture.

2. Process according to claim 1, characterised in that the microreactor is a miniaturised flow reactor.

3. Process according to claim 1 or 2, characterised in that the microreactor is a static micromixer.

4. Process according to one of claims 1 to 3, characterised in that the microreactor is connected via an outlet to a capillary, preferably a heatable capillary.

5. Process according to one of claims 1 to 4, characterised in that the volume of the microreactor is ≦100 μl, preferably ≦50 μl.

6. Process according to one of claims 1 to 5, characterised in that the microreactor is heatable.

7. Process according to one of claims 1 to 6, characterised in that the microreactor has channels having a diameter of from 10 to 1000 μm, preferably from 20 to 800 μm, particularly preferably from 30 μm to 400 μm.

8. Process according to one of claims 1 to 7, characterised in that the reaction mixture flows through the microreactor at a flow rate of from 0.01 μl/min to 100 ml/min, preferably from 1 μl/min to 1 ml/min.

9. Process according to one of claims 1 to 8, characterised in that the residence time of the compounds employed in the microreactor, where appropriate in the microreactor and the capillaries, is from ≦1 second to ≦15 hours, preferably from ≦1 minute to ≦3 hours.

10. Process according to one of claims 1 to 9, characterised in that it is carried out at a temperature of from −100 to +250° C., preferably from −78 to +150° C., particularly preferably from 0° C. to +40° C.

11. Process according to one of claims 1 to 10, characterised in that the course of the reaction is followed by chromatography, preferably by gas chromatography, and if necessary regulated.

12. Process according to one of claims 1 to 11, characterised in that the oxidised carbonyl compound is isolated from the reaction mixture by extraction or precipitation.

13. Process according to one of claims 1 to 12, characterised in that the oxidant employed is at least one oxidant selected from the group consisting of inorganic and organic peroxides, hydrogen peroxide, hydrogen peroxide/urea adduct, peroxo complexes of transition metals, mixtures of peroxo compounds with organic acids and/or inorganic acids and/or Lewis acids, organic peracids, inorganic peracids or dioxiranes, or a mixture of these oxidants.

14. Process according to claim 13, characterised in that the inorganic peroxide employed is an ammonium peroxide, an alkali metal peroxide, preferably sodium peroxide, an ammonium persulfate, an alkali metal persulfate, an ammonium perborate, an alkali metal perborate, an ammonium percarbonate, an alkali metal percarbonate, an alkaline-earth metal peroxide or zinc peroxide, or a mixture of these compounds.

15. Process according to claim 13, characterised in that the organic peroxide employed is tert-butyl hydroperoxide, cumene hydroperoxide, menthyl hydroperoxide, 1-methylcyclohexane hydroperoxide or a mixture of these compounds.

16. Process according to claim 13, characterised in that the peroxo complex of transition metals employed is a peroxo complex of iron, manganese, vanadium or molybdenum or a mixture of these peroxo complexes.

17. Process according to claim 13, characterised in that the peroxo compound with an inorganic acid is potassium peroxodisulfate with sulfuric acid, and the peroxo compound with a Lewis acid is hydrogen peroxide with boron trifluoride.

18. Process according to claim 13, characterised in that the organic peracid employed is perbenzoic acid, m-chloroperbenzoic acid, magnesium monoperphthalic acid, peracetic acid, peroxytrifluoroacetic acid or a mixture of these peracids.

19. Process according to one of claims 1 to 18, characterised in that the organic carbonyl compound employed is an aliphatic, cycloaliphatic, aromatic or heteroaromatic ketone, preferably acetone, cyclohexanone, cyclopentanone or butanone.

20. Process according to one of claims 1 to 19, characterised in that the molar ratio between the organic carbonyl compound and the oxidant is from 1:10 to 1:5, preferably from 1:2 to 1:1.5 and particularly preferably from 1:1 to 1:1.2.
